(19) United States
(12) Patent Application Publication
(10) Pub. No.: US 2026/0345585 A1
(43) Pub. Date: Nov. 5, 2026

(54) DIETARY SUPPLEMENT COMPRISING A PREBIOTIC AND ANTIOXIDANT

(71) Applicant: N.V. Nutricia, Zoetermeer (NL)

(72) Inventors: Yi Wang, Shanghai (CN); Jiangyi Mao, Shanghai (CN); Xianfeng Zhao, Shanghai (CN)

(73) Assignee: N.V. Nutricia, Zoetermeer (NL)

(21) Appl. No.: 19/709,010

(22) Filed: Jun. 16, 2026

Related U.S. Application Data
(63) Continuation of application No. PCT/CN2023/141355, filed on Dec. 25, 2023.

(51) Int. Cl. A61K 31/4745; A61K 9/00; A61K 38/06; A61K 47/36; A61K 47/46

(52) U.S. Cl. CPC A61K 31/4745; A61K 9/0095; A61K 38/063; A61K 47/36; A61K 47/46

(57) ABSTRACT

The present invention relates to an oral composition comprising a prebiotic and an antioxidant. This nutritional composition may be used to support the immune system or to reduce oxidative stress.

### TECHNICAL FIELD OF THE INVENTION

The present invention is in the field of dietary supplements. The present invention relates to an oral composition comprising a prebiotic and an antioxidant. This nutritional composition may be used to support the immune system or to reduce oxidative stress.

### BACKGROUND OF THE INVENTION

The global population is rapidly aging. Today—for the first time in human history—there are more adults over 65 years of age than children under five. From childhood to adulthood, the immune system develops and increases in strength, learning from exposure to infectious agents and building immune memory that enables the body to fight off infections more easily.

Immunosenescence is the gradual deterioration of the immune system, brought on by natural age advancement. The adaptive immune system is affected more than the innate immune system. Immunosenescence involves both the host's capacity to respond to infections and the development of long-term immune memory. The ultimate goal of research related to immunosenescence is to develop strategies to stimulate immunity in the elderly. The aim is to increase health span, defined as years of healthy living.

Oxidative stress reflects an imbalance between the systemic manifestation of reactive oxygen species (ROS) and a biological system's ability to readily detoxify the reactive intermediates or to repair the resulting damage. In humans, oxidative stress is thought to be involved in the development of several diseases such as cancer, neurodegenerative diseases, cardiovascular diseases and infection.

Beta-glucans comprise a group of β-D-glucose polysaccharides (glucans) naturally occurring in the cell walls of cereals, bacteria, and fungi, with significantly differing physicochemical properties dependent on source. The most common forms of β-glucans are those comprising D-glucose units with β-1,3 links. Yeast and fungal β-glucans contain β-1-6 side branches, while cereal β-glucans contain both β-1,3 and β-1,4 backbone bonds. Differences in molecular weight, shape, and structure of β-glucans dictate the differences in biological activity.

Pyrroloquinoline quinone (PQQ, CAS number 72909-34-3), an aromatic tricyclic water soluble quinone, was identified initially as a redox cofactor for bacterial dehydrogenases. PQQ is an antioxidant that occurs naturally in plant foods and animals, however only bacteria are capable of synthesizing PQQ.

Glutathione (GSH, CAS number 70-18-8) is a tripeptide and acts as an antioxidant. Humans are capable of synthesizing GSH.

WO2014007635A1 describes a dietary fibre for use in delaying the satiating effect of medical nutrition or a medicament. In the examples a composition is described comprising PQQ and a cereal fibre, comprising >1 wt. % beta-glucan.

JP2014084302 describes a method of stabilizing S-adenosylmethionine, by providing a composition comprising S-adenosylmethionine-containing yeast and pyrroloquinoline quinone.

There is a need in the art for oral compositions which stimulate the immune system and reduce oxidative stress, as consumption of such compositions contributes to healthy aging and postponement of age-related health problems.

### SUMMARY OF THE INVENTION

The inventors of the present invention performed in vitro experiments with macrophages under stress (LPS—induced) in order to identify beta-glucans that have a strong effect on immunity. Yeast beta-glucans having a backbone of D-glucose monomers linked by a β-1,3 glycosidic bond and side-chains of D-glucose monomers linked by a β-1,6 glycosidic bond, preferably with a ratio of β-1,3 to β-1,6 glycosidic bond of at least 2:1, provided a strong effect on immunity.

The inventors have surprisingly found that the combination of a yeast beta-glucan and PQQ has a beneficial synergistic effect on the immune response and the reduction of oxidative stress. In addition, the inventors found that the combination of yeast beta-glucan, PQQ and GSH also results in a beneficial synergistic effect on the immune response and the reduction of oxidative stress.

The invention thus pertains to a powdered oral composition comprising by weight of dry matter:
i. at least 0.1 wt. % of yeast beta-glucan having
   a molecular weight of at least 5 kDa;
   a backbone of D-glucose monomers linked by a β-1,3 glycosidic bond; and
   side-chains of D-glucose monomers linked by a β-1,6 glycosidic bond;
ii. at least 0.01 wt. % of pyrroloquinoline quinone (PQQ);
wherein the weight ratio of beta-glucan to PQQ is between 40:1-1:10.

When the yeast beta-glucan is combined with PQQ (and optionally GSH) in lower concentrations, this resulted in stronger effects, compared to the same ingredients as such at a higher concentration, which is indicative of synergy between the ingredients.

The invention further relates to the use of yeast beta-glucan and pyrroloquinoline quinone in the manufacture of an oral composition for:
a. improving the immune response in a human subject; and/or
b. reducing oxidative stress in a human subject;
wherein the oral composition comprises by weight of dry matter:
i. at least 0.1 wt. % of yeast beta-glucan having
   a molecular weight of at least 5 kDa;
   a backbone of D-glucose monomers linked by a β-1,3 glycosidic bond; and
   side-chains of D-glucose monomers linked by a β-1,6 glycosidic bond;
ii. at least 0.01 wt. % of pyrroloquinoline quinone (PQQ);
wherein the weight ratio of beta-glucan to PQQ is between 40:1-1:10.

### DETAILED DESCRIPTION OF THE INVENTION

A first aspect of the invention thus pertains to a powdered oral composition comprising by weight of dry matter:
i. at least 0.1 wt. % of yeast beta-glucan having
   a molecular weight of at least 5 kDa;
   a backbone of D-glucose monomers linked by a β-1,3 glycosidic bond; and
   side-chains of D-glucose monomers linked by a β-1,6 glycosidic bond;
ii. at least 0.01 wt. % of pyrroloquinoline quinone (PQQ);
wherein the weight ratio of beta-glucan to PQQ is between 40:1-1:10.

The powdered oral composition preferably comprises at most 10 wt. % water, more preferably at most 7.5 wt. % water and most preferably the oral composition comprises 0-5 wt. % water.

The oral composition is preferably packaged in a sachet.

Preferably the oral composition is packaged in single-dosage units, more preferably single-dosage units of 0.5-40 grams, most preferably single-dosage units of 5-15 grams.

The oral composition may be suitably reconstituted with water or another food grade aqueous liquid to form a ready-to drink composition in liquid form. Preferably 5-20 grams of this oral composition is added to 50-200 ml of liquid to arrive at a ready-to-drink liquid.

Preferably the oral composition comprises 0.2-50 wt. % of yeast beta-glucan by weight of dry matter, more preferably 0.5-25 wt. % of yeast beta-glucan and most preferably 1-10 wt. % of yeast beta-glucan.

Preferably, the yeast beta-glucan is purified yeast beta-glucan. Preferably the oral composition does not comprise intact yeast cells.

Preferably the yeast beta-glucan is purified from yeast by methods known to the skilled person in the art. Extraction of beta-glucan from yeast typically comprises two major steps, the first being yeast cell lysis, the second being the separation of beta-glucan from the other cell wall components. This may be done by chemical methods (high acid, high base), physical methods (sonication, high pressure) or enzymatic methods. Subsequently the separated beta-glucan may be further purified and/or dried to a beta-glucan powder

Preferably the yeast beta-glucan is beta-glucan from Saccharomyces cerevisiae, also known as brewer's yeast or baker's yeast.

Preferably the yeast beta-glucan is not soluble in water.

Preferably the yeast beta-glucan has a molecular weight of between 10-5,000 kDa, preferably 25-2,000 kDa, more preferably 50-1,000 kDa.

Preferably the yeast beta-glucan has a Mn (number average molar mass) of between 5-1,000 kDa, preferably 10-500 kDa, more preferably 20-250 kDa.

Preferably the ratio between the β-1,3 glycosidic bond and the β-1,6 glycosidic bond in the yeast beta-glucan is at least 2:1, more preferably between 2.5:1-5:1.

Preferably the oral composition comprises 0.02-40 wt. % of PQQ by weight of dry matter, more preferably 0.04-20 wt. % PQQ and most preferably 0.06-10 wt. % PQQ.

The PQQ is preferably prepared by bacterial fermentation and may be suitable sourced from commercial companies such as Shandong Jincheng Biopharmaceutical Co. Ltd.

Preferably, the weight ratio of yeast beta-glucan to PQQ in the oral composition is between 20:1 to 1:1, preferably between 15:1-5:1.

Preferably the oral composition further comprises at least 0.01 wt. % of glutathione (GSH) by weight of dry matter, more preferably 0.1-50 wt. % GSH and even more preferably 0.5-25 wt. % GSH and most preferably 1-10 wt. % GSH.

Preferably, the weight ratio of beta-glucan to GSH is between 400:1 and 1:1, more preferably between 300:1 and 50:1 and most preferably 200:1 and 100:1.

In a preferred embodiment the oral composition further comprises milk powder. More preferably the oral composition comprises at least 50 wt. % milk powder by weight of dry matter, most preferably the oral composition comprises 60-98 wt. % of milk powder.

Preferably the milk powder is derived from cow's milk, goat milk, sheep milk or combinations thereof, more preferably the milk powder is derived from cow's milk.

Preferably the milk powder is a blend of whole milk powder and skimmed milk powder, more preferably the milk powder is a blend of whole milk powder and skimmed milk powder in a weight ratio between 10:1 to 1:10, even more preferably in a weight ratio between 5:1 to 1:5, and most preferably in a weight ratio between 2:1 to 1:2.

In a particularly preferred embodiment, the oral composition comprises by weight of dry matter:
0.2-10 wt. % yeast beta glucan;
0.02-2 wt. % PQQ;
0-2 wt. % GSH; and
70-98 wt. % milk powder.

In alternative preferred embodiment, the oral composition comprises by weight of dry matter:
2-50 wt. % beta glucan;
0.2-10 wt. % PQQ;
0-10 wt. % GSH; and
49-98 wt. % food-grade carrier.

Suitable examples of food-grade carriers are inert powdered substances such as starch, maltodextrin, cellulose, fructose, or mannitol.

Preferably, the oral composition further comprises non-digestible oligosaccharides, more preferably prebiotic non-digestible oligosaccharides.

Preferably, the oral composition comprises 0.1-20 wt. % non-digestible oligosaccharides by weight of dry matter, even more preferably 0.2-10 wt. % non-digestible oligosaccharides and most preferably 0.5-5 wt. % non-digestible oligosaccharides.

Preferably the non-digestible oligosaccharides are selected from fructo-oligosaccharide (FOS), galacto-oligosaccharides (GOS), polydextrose and combinations thereof. More preferably, the non-digestible oligosaccharides are selected from FOS, GOS and combinations thereof.

The GOS preferably are beta-galacto-oligosaccharides. Beta-galacto-oligosaccharides may also be referred to as trans-galacto-oligosaccharides (TOS). GOS is for example sold under the trademark Vivinal™ (Borculo Domo Ingredients, Netherlands) or Bi2Munno (Classado). The GOS preferably has a DP of 2-60, more preferably a DP of 3-40, even more preferably a DP of 4-20 and most preferably a DP of 5-10.

FOS preferably comprises a chain of beta-linked fructose units with a DP of 2 to 250, more preferably 5 to 100, even more preferably 10 to 60. A FOS suitable for use in the compositions is also commercially available, e.g. Raftiline®HP (Orafti).

A second aspect of the invention relates to the use of yeast beta-glucan and pyrroloquinoline quinone in the manufacture of an oral composition for:
a. improving the immune response in a human subject; and/or
b. reducing oxidative stress in a human subject;
wherein the oral composition comprises by weight of dry matter:
i. at least 0.1 wt. % of yeast beta-glucan having
   a molecular weight of at least 5 kDa;
   a backbone of D-glucose monomers linked by a β-1,3 glycosidic bond; and
   side-chains of D-glucose monomers linked by a β-1,6 glycosidic bond;
ii. at least 0.01 wt. % of pyrroloquinoline quinone (PQQ).
wherein the weight ratio of beta-glucan to PQQ is between 40:1-1:10.

Preferably, the embodiments as described herein above in relation to the first aspect of the invention equally apply to the second aspect of the invention.

Preferably, the oral composition is a powdered oral composition.

Preferably, the human subject is at least 40 years old, more preferably 50-100 years old.

Preferably, the oral composition is administered at least once daily for a period of at least 3 days, more preferably at least daily for a period of at least 2 weeks, most preferably at least daily for a period of at least 4 weeks.

Preferably, “improving the immune response in a human subject” and “reducing oxidative stress in a human subject” are compared to another human subject who did not consume the oral composition of the invention, in other words compared to another human subject who did not consume an oral composition comprising a combination of yeast beta-glucan, PQQ and GSH.

Preferably the improvement of the immune response results in the human subject having less infectious disease, more preferably results in the human subject having less disease due to viral or bacterial infections, more preferably results in the human subject having less disease due to food-borne disease, corona virus, rhinovirus, respiratory syncytial virus or influenza.

Preferably the improvement of the immune response results in the human subject recovering sooner after falling ill with a viral or bacterial infection, more preferably results in the human subject recovering sooner after falling ill due to food-borne disease, corona virus, rhinovirus, respiratory syncytial virus or influenza.

Preferably, the improved immune response is by an increased macrophage response.

The term “oxidative stress”, as used herein, refers to a state of imbalance between oxidation and antioxidation in the body and is capable of causing damage to various cellular constituents, including DNA and tissues, leading to oxidative stress damage and, ultimately, oxidative stress-mediated disorders.

Preferably, the reduction in oxidative stress results in the human subject having a lower risk of developing oxidative stress-mediated disorders, preferably oxidative stress-mediated disorders selected from cancer, neurodegenerative diseases, cardiovascular diseases and infection.

Preferably, the reduced oxidative stress is by reduced reactive oxygen species production.

Preferably, the reduced oxidative stress contributes to healthy aging.

In some jurisdictions, the present invention may be defined as an oral composition comprising by weight of dry matter:
i. at least 0.1 wt. % of yeast beta-glucan having
   a molecular weight of at least 5 kDa;
   a backbone of D-glucose monomers linked by a β-1,3 glycosidic bond; and
   side-chains of D-glucose monomers linked by a β-1,6 glycosidic bond;
ii. at least 0.01 wt. % of pyrroloquinoline quinone (PQQ).
wherein the weight ratio of beta-glucan to PQQ is between 40:1-1:10;
for use in:
   a. improving the immune response in a human subject; and/or
   b. reducing oxidative stress in a human subject.

In some jurisdictions, the present invention may be defined as a method for:
a. improving the immune response in a human subject; and/or
b. reducing oxidative stress in a human subject;
said method comprising administering to said subject an oral composition comprising by weight of dry matter:
i. at least 0.1 wt. % of yeast beta-glucan having
   a molecular weight of at least 5 kDa;
   a backbone of D-glucose monomers linked by a β-1,3 glycosidic bond; and
   side-chains of D-glucose monomers linked by a β-1,6 glycosidic bond;
ii. at least 0.01 wt. % of pyrroloquinoline quinone (PQQ).
wherein the weight ratio of beta-glucan to PQQ is between 40:1-1:10.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The present invention is illustrated by the following non-limiting examples.

### EXAMPLES

### Macrophage Assay

RAW264.7 macrophages were purchased from the National Collection of Authenticated Cell Cultures (Shanghai, China). The cell culture medium was supplemented with 10% fetal bovine serum and 100 U/mL penicillin and 100 μg/mL streptomycin in DMEM culture medium.

### Macrophage Proliferation

The viability of RAW264.7 cells was measured by Cell Counting Kit (CCK-8). Briefly, RAW264.7 cells were plated into 96-well plates at a density of 5×10⁴ cells/well and incubated at 37 °C, 5% CO₂ for 24 hours. Next the cells were exposed to 1 μg/mL lipopolysaccharides (LPS) for 24 hours to induce an inflammatory response by the RAW264.7 cells. Subsequently, the cells were exposed to different combinations of test ingredients for 24 hours. For the control, the cells were stimulated with LPS for 24 hours, followed by 24 hours with medium only. All experiments were performed in triplicate. Next, the cells were incubated with 10 μL/mL CCK-8 reagent for 2 hours. The absorbance was recorded on an automatic microplate reader with an excitation wavelength of 450 nm. The higher the measured value, the higher the number of viable cells.

### ROS Production

The accumulation of ROS was assessed by the radical probe DCFH-DA. At the beginning, RAW264.7 cells were seeded in 6-well plate at 1×10⁶ cells/well for 24 hours followed by a 24 hour exposure to LPS to induce an inflammatory response by the RAW264.7 cells. Subsequently, the cells were exposed to different combinations of test ingredients. For the control, the cells were stimulated with LPS for 24 hours, followed by 24 hour with medium only. All experiments were performed in triplicate. Next, 10 UL DCFH-DA, diluted 1:1000 with serum-free medium, was added to the wells, after removing all the cell culture medium. After incubation at 37 °C for 20 minutes in the dark, the cells were washed and lysed to release intracellular DCF and the fluorescence intensity was detected at 488 nm excitation and 525 nm emission using a microplate spectrofluorometer. The higher the measured value, the higher the level of ROS.

### Example 1

Four different yeast beta-glucans were compared for their effect on macrophage proliferation. The tested yeast beta-glucans are described in Table 1. These yeast beta-glucan were purchased in dried form from commercial suppliers and were all derived from Saccharomyces cerevisiae.

The ratio of the-1,3 to β-1,6 glycosidic bond was determined by nuclear magnetic resonance. The Mw and Mn was determined by high performance liquid phase exclusion chromatography.

TABLE 1

| Yeast | Properties |
|---|---|
| A | Ratio β-1,3 to β-1,6 glycosidic bond: 4:1 |
| | Mw (weight average molecular weight): 800 kDa |
| | Mn (number average molecular weight): 47 kDa |
| | Insoluble |
| B | Ratio β-1,3 to β-1,6 glycosidic bond: 2.7:1 |
| | Mw (weight average molecular weight): 1300 kDa |
| | Mn (number average molecular weight): 180 kDa |
| | Insoluble |
| C | Ratio β-1,3 to β-1,6 glycosidic bond: 2:1 |
| | Mw (weight average molecular weight): 5-960 kDa |
| | Mn (number average molecular weight): 2-520 kDa |
| | Water-soluble |
| D | Ratio β-1,3 to β-1,6 glycosidic bond: 3.9:1 |
| | Mw (weight average molecular weight): 900 kDa |
| | Mn (number average molecular weight): 14 KDa |
| | Insoluble |

### Results

The macrophage proliferation is expressed as a percentage compared to the control (LPS only) which was set to 100%. A higher macrophage response was considered beneficial as it is an indication that a stronger immune response against the LPS trigger was generated. The results for the different yeast beta-glucans are shown in Table 2.

TABLE 2

| Yeast beta-glucan | 100 μg/ml | 200 μg/ml |
|---|---|---|
| A | 181.9 ± 5.1 | 217.8 ± 17.6 |
| B | 144.8 ± 20.5 | 164.5 ± 14.8 |
| C | 117.1 ± 9.4 | 107.5 ± 9.8 |
| D | 104.4 ± 7.3 | 116.9 ± 10.0 |

Yeast beta-glucan A and B showed the strongest effect on macrophage proliferation.

### Example 2

The tested ingredients are described in Table 3

TABLE 3
Ingredient

Yeast beta-glucan A (Table 1)
Yeast beta-glucan B (Table 1)
Pyrroloquinoline quinone (PQQ)
Glutathione (GSH) in a yeast extract
Effective GSH content: 12%

### Collaborative Evaluation

For this evaluation, the concentration at which each test ingredient provided an equivalent effect on macrophage proliferation was determined. Concentrations that provided an equivalent effect are listed in Table 4.

TABLE 4

| | Concentration (μg/mL) |
|---|---|
| β-glucan | 100 |
| PQQ | 10 |
| GSH | 5 μg/mL yeast extract |
| | 0.6 μg/mL GSH |

A combination of two compounds was considered as having synergy if both values, as calculated according to formulas 1a and 1b below, were higher than 1. In the formula's below the “a” refers to the concentration of the 1^{st} compound and “b” to the concentration of the 2^{nd} compound. If a combination of three compounds were to be evaluated, a similar logic is applied, but the assessed concentration in the combination is divided by 3 and three comparisons are made.

$$\frac{E ( \frac{a}{2} + \frac{b}{2} )}{E ( a )} \qquad ( 1 a )$$

$$\frac{E ( \frac{a}{2} + \frac{b}{2} )}{E ( b )} \qquad ( 1 b )$$

### Results-Beta-Glucan+PQQ

The macrophage proliferation and ROS production are expressed as a percentage compared to the control (LPS only) which was set to 100%. A higher macrophage response was considered beneficial as it is an indication that a stronger immune response against the LPS trigger was generated. A lower ROS production was considered beneficial as less ROS results in less cell damage due to lower oxidative stress. The results for the combination of beta-glucan and PQQ are shown in Table 5 and 6.

TABLE 5
Macrophage proliferation

| | | | β-glucan |
|---|---|---|---|
| | (a) β-glucan A | (b) PQQ | A + PQQ |
| Concentration (μg/mL) | 100 | 10 | 50 + 5 |
| Macrophage proliferation | 158.0 ± 5.3 | 142.0 ± 14.1 | 183.8 ± 8.1 |
| (%) | | | |
| Formula 1a | — | — | 1.2 |
| Formula 1b | — | — | 1.3 |

TABLE 6
ROS production

| | | | β-glucan |
|---|---|---|---|
| | (a) β-glucan A | (b) PQQ | A + PQQ |
| Concentration (μg/mL) | 100 | 10 | 50 + 5 |
| ROS production (%) | 53.3 ± 1.3 | 62.8 ± 5.3 | 35.9 ± 2.9 |
| Delta to Control (%) | 46.7 ± 1.3 | 37.2 ± 5.3 | 64.1 ± 2.9 |
| Formula 1a (using delta) | — | — | 1.4 |
| Formula 1b (using delta) | — | — | 1.7 |

### Results—Beta-Glucan+PQQ+GSH

The results for the combination of beta-glucan, PQQ and GSH are shown in Table 7 and 8.

TABLE 7
Macrophage proliferation

| | (a) β- | | | β-glucan B + |
|---|---|---|---|---|
| | glucan B | (b) PQQ | (c) GSH | PQQ + GSH |
| Concentration | 100 | 10 | 0.6 (5) | 33 + 3.3 + |
| (μg/mL) | | | | 0.2 (1.67) |
| Macrophage | 140.1 ± 24.1 | 142.0 ± 14.1 | 141.8 ± 14.5 | 174.3 ± 11.3 |
| proliferation | | | | |
| (%) | | | | |
| Formula 1a | — | — | — | 1.2 |
| Formula 1b | — | — | — | 1.2 |
| Formula 1c | — | — | — | 1.2 |

TABLE 8
ROS production

| | (a) β- | | | β-glucan B + |
|---|---|---|---|---|
| | glucan B | (b) PQQ | (c) GSH | PQQ + GSH |
| Concentration (μg/mL) | 100 | 10 | 0.6 (5) | 33 + 3.3 + |
| | | | | 0.2 (1.67) |
| ROS production (%) | 58.1 ± 4.2 | 62.8 ± 5.3 | 51.1 ± 4.2 | 41.2 ± 9.9 |
| Delta to Control (%) | 41.9 ± 4.2 | 37.2 ± 5.3 | 48.9 ± 4.2 | 58.8 ± 9.9 |
| Formula 1a (using | — | — | — | 1.4 |
| delta) | | | | |
| Formula 1b (using | — | — | — | 1.6 |
| delta) | | | | |
| Formula 1c (using | — | — | — | 1.2 |
| delta) | | | | |

### Example 3

A sachet comprising 10 grams of powdered composition. The powder is to be dissolved in 100 ml of water before consumption.

| Ingredients | Wt. % by weight of dry matter |
|---|---|
| Yeast beta-glucan | 5 |
| PQQ | 0.5 |
| GSH | 0.05 |
| Milk powder | 94.45 |

1. A powdered oral composition comprising by weight of dry matter:
i. at least 0.1 wt. % of yeast beta-glucan having
a molecular weight of at least 5 kDa;
a backbone of D-glucose monomers linked by a β-1,3 glycosidic bond; and
side-chains of D-glucose monomers linked by a β-1,6 glycosidic bond;
ii. at least 0.01 wt. % of pyrroloquinoline quinone (PQQ);
wherein the weight ratio of beta-glucan to PQQ is between 40:1-1:10.

2. The oral composition according to claim 1, wherein the yeast beta-glucan has a molecular weight between 10-5000 kDa.

3. The oral composition according to claim 1, wherein the ratio between the β-1,3 glycosidic bond and the β-1,6 glycosidic bond in the yeast beta-glucan is at least 2:1.

4. The oral composition according to claim 1, wherein the weight ratio of beta-glucan to PQQ is between 20:1 to 1:1.

5. The oral composition according to claim 1, wherein the oral composition further comprises at least 0.01 wt. % of glutathione (GSH) by weight of dry matter.

6. The oral composition according to claim 5, wherein the weight ratio of beta-glucan to GSH is between 400:1 and 1:1.

7. The oral composition according to claim 1, wherein the oral composition further comprises at least 50 wt. % milk powder by weight of dry matter.

8. The oral composition according to claim 7, wherein the milk powder is a blend of whole milk powder and skimmed milk powder.

9. The oral composition according to claim 1, wherein the oral composition comprises by weight of dry matter:
0.2-10 wt. % yeast beta glucan;
0.02-2 wt. % PQQ;
0-2 wt. % GSH; and
70-98 wt. % milk powder.

10. The oral composition according to claim 1, wherein the oral composition comprises by weight of dry matter:
2-50 wt. % beta glucan;
0.2-10 wt. % PQQ;
0-10 wt. % GSH; and
49-98 wt. % food-grade carrier.

11. The oral composition according to claim 1, wherein the composition further comprises non-digestible oligosaccharides, preferably one or more non-digestible oligosaccharides selected from fructo-oligosaccharides, galacto-oligosaccharides or polydextrose.

12. The oral composition according to claim 11, wherein the composition comprises 0.1-20 wt. % non-digestible oligosaccharides by weight of dry matter.

13.-15. (canceled)

16. A method for
a) improving the immune response in a human subject; and/or
b) reducing oxidative stress in a human subject
the method comprising administering to the human subject in need thereof the oral composition according to claim 1.

17. The method according to claim 16, wherein the human subject is at least 40 years old.

18. The method according to claim 16, wherein the oral composition is administered at least once daily for a period of at least 3 days.
